(19) United States
(12) Patent Application Publication
(10) Pub. No.: US 2013/0253060 A1
(43) Pub. Date: Sep. 26, 2013

(54) Intramural Research Program of the National Institutes of Health, National Institute of Environmental Health Sciences (NIEHS)

(71) Applicant: Harihara M. Mehendale, West Monroe, LA (US)

(72) Inventor: Harihara M. Mehendale, West Monroe, LA (US)

(21) Appl. No.: 13/613,606

(22) Filed: Sep. 13, 2012

Related U.S. Application Data
(60) Provisional application No. 61/537,299, filed on Sep. 21, 2011.

(51) Int. Cl. A61K 31/197

(52) U.S. Cl. CPC A61K 31/197
USPC 514/563

(57) ABSTRACT

Use of sPLA₂ inhibitors (for example, BPPA (5-(4-Benzyloxyphenyl)-4S-(7-phenylheptanoylamino) pentanoic acid)) within about 9 hours after poisoning with a liver toxin, for example, acetaminophen (APAP), was shown to increase survivorship. The inhibition of sPLA₂ lead to markedly decreased progression of liver injury as reflected in lower alanine aminotransferase (ALT—a biomarker for liver injury) levels, and to significantly higher survival rates. Similar treatment in human patients suffering from hepatotoxicity will be effective in increasing survival, and treatment with other sPLA2 inhibitors will also be effective in decreasing liver damage and mortality.

The benefit of the filing date of provisional U.S. application Ser. No. 61/537,299, filed 21 Sep. 2011, is claimed under 35 U.S.C. §119(e).

This invention was made with government support under an Intramural Research Program of the National Institutes of Health, National Institute of Environmental Health Sciences (NIEHS). The government has certain rights in the invention.

This invention pertains to use of inhibitors of secretory phospholipase A₂ (sPLA₂) to reduce mortality and hepatoxicity from exposure to high levels of hepatotoxins, for example, acetaminophen (APAP).

Toxicant-Induced Tissue Injury

Upon biotransformation, toxicants may be bioactivated to reactive metabolites and organic free-radicals or be converted to innocuous products amenable for elimination. Reactive metabolites covalently bind to tissue macromolecules, disrupting cellular functions such as osmoregulation and homeostasis. Other toxicants may opportunistically enter crucial metabolism pathways to block energy needed to drive physiological functions. Individually or collectively, these events are known to cause cell death, which continues while the parent toxicant or the metabolites remain in the target tissue.

Research has shown that injury continues to spread even after the initial toxicant and its metabolites have been eliminated after exposure to a lethal dose (Chilakapati et al., 2007a; Chilakapati et al., 2007b; Mangipudy et al., 1995). For example, thioacetamide (TA), a model hepatotoxicant, has shown this property in rats. Liver injury measured as an increase in plasma alanine transaminase (ALT) continued even after 24 h (12 half-lives of TA), indicating that progression of liver injury occurs even after all TA is eliminated. This indicates that events other than TA or thioacetamide sulfoxide (TASO), its metabolite, were responsible for continued progression of liver injury (Chilakapati et al., 2007a).

Systematic studies to address how toxicant-initiated tissue injury progresses are scant. Progression of liver injury mediated by the cytoplasmic, lysosomal and other hydrolytic enzymes, “death proteins”, has been proposed. These death proteins are normally contained inside cells under tight regulation by micromolar Ca²⁺ concentration. They become activated upon release into the extracellular fluid from oncotically necrosed and lysed hepatocytes and exposure to the high (1.3 mM) Ca²⁺ concentration present outside the cells (extracellularly) [7-9]. The activated death proteins hydrolyze their substrates in the plasma membrane of neighboring cells, commencing a self-perpetuated injury progression.

Acetaminophen Poisoning

Acetaminophen (APAP; for example, TYLENOL®) is a widely used analgesic and antipyretic drug. At high doses its toxic metabolite, N-acetyl-p-benzoquinone imine (NAPQ1), depletes glutathione (GSH) and causes liver necrosis, liver failure, and death. With a half-life of 1 h in mice, no APAP is expected to remain in the body after eight hours (Shankar et al., 2003). The initiated injury continues to expand even after the offending chemical and its metabolites are eliminated from the body (Mangipudy et al., 1995; Shankar et al., 2003).

Hundreds of patients die every year from an overdose of acetaminophen (“APAP”; e.g. TYLENOL®). Current treatment for the life-threatening acetaminophen overdose is intravenous administration of N-acetylcysteine (NAC) to tie up the NAPQ1 toxic metabolite of acetaminophen. After further metabolism, the complex is excreted in urine. To be effective, the NAC treatment must be administered within a few hours after overdosing. Early treatment is critical because the reactive metabolite of APAP covalently binds to liver tissue and initiates liver necrosis unless it is trapped by NAC. However, NAC treatment is not effective if postponed several hours after overdosing. Because overdosed patients are most often not discovered until after several hours have lapsed, NAC treatment is often ineffective in saving lives. Other problems with NAC treatment for acetaminophen overdose are that the NAC is recommended to be given multiple times per day every 4 hours, and the NAC is no longer effective after blood levels of acetaminophen fall below detectable levels. Liver injury continues to escalate for several days even though acetaminophen can no longer be detected in the body, and continuing progression of liver necrosis causes the liver to fail which leads to death. NAC is completely ineffective in stopping progression/expansion of injury. It is only effective as long as there is acetaminophen in the liver to be bioactivated. The half-life of acetaminophen in humans is 2.5 hours. In about 7 half-lives, 99% of the drug would have been eliminated from the body. Nevertheless, the lethal overdosing still leads to mortality.

Death Proteins and Progression of Injury

A novel and less studied mechanism to explain the progression of injury is the spillage of hydrolytic enzymes from the dying cells into the extracellular Ca²⁺-rich environment, which then destroy the neighboring partly affected or healthy cells. Progression of liver injury mediated by the cytoplasmic and lysosomal hydrolytic enzymes spilt from oncotically necrosed hepatocytes has been proposed (Limaye et al., 2003; Mehendale et al., 2008). These enzymes, activated by the extracellular high Ca²⁺ (1.3 mM), attack the neighboring hepatocytes (Rynviak et al., 1990; Hayami et al., 1999) and mediate the progression of tissue injury. This is supported by a report that thioacetamide (TA)-induced apoptosis is the result of caspase-3 released from necrotic hepatocytes rather than due to a direct effect of TA (Rynviak et al., 1990; Hayami et al., 1999), and is consistent with the notion that various hydrolytic enzymes spilt from necrosed hepatocytes drive progression of tissue injury (Limaye et al., 2003; Mehendale et al., 2008).

Death proteins are enzymes that spill out into the intercellular spaces in tissues as a result of cellular necrosis. Inside the cells, their natural habitat, death proteins are highly regulated by micromolar concentrations of calcium and are harmless. However, once they are outside the cells, the extracellular high calcium (1.3 mM) activates the hydrolytic enzymes such as proteases and phospholipases normally contained in cell cytosol. Once activated, the hydrolytic enzymes hydrolyze their respective substrates contained in the plasma membranes of the surrounding healthy or partially affected cells. As a result, necrotic cell death continues causing an unabated progression of injury mediated by hydrolytic degradative enzymes, known as death proteins, such as calpain, phospholipases, and nucleases (Limaye et al., 2003; Limaye et al., 2006; Napirei et al., 2006; Bhave et al., 2008a; Bhave et al., 2008b). Regression of injury is accompanied by structural and functional restoration by replacement of lost and damaged tissue accompanied by resolution of inflammation. Facilitative role of cyclooxygenase-2 (COX-2) has been established in liver regeneration in diverse models of liver injury such as partial hepatectomy, drug- or toxicant-induced hepatotoxicity, and liver disease (Ishida et al., 2004; Barbuio et al., 2007; Malleo et al., 2007). Secretory phospholipase A2 (sPLA2) along with other death proteins, was shown to mediate progression of liver injury initiated by CCl₄, but in the absence of concomitantly induced COX-2, liver injury expanded and progressed unabatedly. The ratio of COX-2 to sPLA2 activities provided a predictive measure of the net effect on hepatotoxicity (Bhave et al., 2008a; Bhave et al., 2008b).

Secretory Phospholipase A₂ (sPLA₂)

sPLA₂ spills out of cells in many effected tissues including the necrosed hepatocytes, and is secreted in the extracellular space following drug/toxicant-initiated injury (Limaye et al., 2003; Mehendale et al., 2008). sPLA₂ hydrolyzes the ester bond at the sn-2 position of glycerophospholipids in the presence of high Ca²⁺ with a broad fatty acid and phospholipid specificity (Wolf et al., 1997). sPLA₂ is expressed in hepatocytes and macrophages at basal levels and is stored in cytosolic granules, or synthesized upon proinflammatory stimulation and then secreted in the intercellular space, its site of action. The physiological functions of sPLA₂ include release of arachidonic acid (AA) from dietary and membrane phospholipids. Plasma and tissue concentrations of sPLA₂ correlate well with the severity of disease in several immune-mediated inflammatory pathologies, such as rheumatoid arthritis, septic shock, psoriasis, Crohn's disease, respiratory distress syndrome, and asthma (Kramer et al., 1989). Activation of sPLA₂ is associated with ischemic injury in rat kidney (Takasaki et al., 1998) and atherosclerosis in sPLA₂ transgenic mice (Ivandic et al., 1999). sPLA₂ has also been implicated in human liver diseases (Poli, 1993), neurodegenerative conditions (Cunningham et al., 2004), colitis (Woodruff et al., 2005), and myocardial ischemia/reperfusion injury (Fujioka et al., 2008). Membrane rearrangement stimulated by proinflammatory cytokines, IL-1, TNF-α, and mitogens is accompanied by markedly induced expression of sPLA₂ (Murakami et al., 1996), an effect downregulated by anti-inflammatory cytokines in a wide variety of cells and tissues.

Plasma sPLA₂ activity has been shown to increase in rats after dosing with CCl₄/kg, and the increase was greater with higher doses of CCL₄, and corresponded to higher liver necrosis and 70% mortality (Bhave et al., 2008a).

Another example of progression of tissue injury mediated by sPLA₂ is ischemia reperfusion injury observed in heart, liver, lung, kidney, and intestine. sPLA₂ mediates the breakdown of membrane phospholipids of various tissues. The release and activation of sPLA₂ in ischemia/reperfusion injury results in breakdown of membrane phospholipids, excessive AA, and the harmful effects of thromboxanes and leukotrienes produced through favorably tipped 5-LO pathway.

Recent work with inhibition of sPLA₂ has shown attenuation of acute cardiogenic pulmonary edema (Kuwabata et al., 2010), treatment of coronary artery disease (Karakas et al., 2009), prevented the progress of CCL₄-induced phosphatidylethanomine hydrolysis (Moon et al. 2008), treatment of hepatocirrhosis (U.S. Pat. No. 6,967,200), and attenuation of LPS-induced acute lung injury in mice (Sato et al., 2010). Several inhibitors are known for sPLA₂, including without limitation, LY329722 (sodium [3-aminooxyalyl-1-benzyl-2-ethyl-6-methyl-1H-indol-4-yloxy]-acetic acid), ochnaflavone (a naturally occurring biflavonoid), varespladib (Dzavik et al., 2010), BPPA (5-(4-benzyloxyphenyl)-4S-(7-phenylhepatonoylamino) pentanoic acid (Arumugam et al., 2003), and p-bromophenacylbromide (p-BPB) and other benzophenone oximes derivatized with syndone (Kamble et al., 2009).

I have discovered that mice treated with an sPLA₂ inhibitor (for example, BPPA (5-(4-Benzyloxyphenyl)-4S-(7-phenylheptanoylamino) pentanoic acid)) 2, 4 or 8 hours after poisoning with the liver toxin, acetaminophen (APAP), yielded 90, 70 and 60% survival, respectively. BPPA prevented progression of liver injury initiated by APAP by inhibiting the death protein, sPLA₂. The inhibition of sPLA₂ leads to markedly decreased progression of liver injury as reflected in lower alanine aminotransferase (ALT—a biomarker for liver injury) levels. Therefore, there is a higher survival rate among the BPPA-treated mice. Decreasing plasma sPLA₂ and ALT activities correspond with higher survival. It is believed that the same effect will be seen in human patients suffering from APAP hepatotoxicity, and that treatment with other sPLA₂ inhibitors will also be effective in decreasing liver damage and mortality.

I have also shown that mice lethally dosed with thioacetamide (data not shown), and mice dosed with chlordecone+carbon tetrachloride had less mortality when treated with BPPA. Thus I believe that inhibition of sPLA₂ can be an effective treatment to protect from hepatoxicity caused by other toxins.

### BRIEF DESCRIPTION OF DRAWINGS

FIG. 1 illustrates the proposed mechanism of lethal liver injury in mammals induced by acetaminophen and subsequent rescue by inhibition of secretory phospholipase A₂.

FIG. 2 illustrates the normal plasma levels of alanine aminotransferase (ALT) and secretory phospholipase A₂ (sPLA₂) over 14 days in control mice.

FIG. 3 illustrates the plasma levels of alanine aminotransferase (ALT) and secretory phospholipase A₂ (sPLA₂) over 14 days in vehicle-treated mice (injected with a single dose of 0.45 N NaCl).

FIG. 4 illustrates the survivorship and the plasma levels of alanine aminotransferase (ALT) and secretory phospholipase A₂ (sPLA₂) over 14 days in mice injected with a single lethal dose (600 mg/kg) of acetaminophen (APAP).

FIG. 5 illustrates the survivorship and the plasma levels of alanine aminotransferase (ALT) and secretory phospholipase A₂ (sPLA₂) over 14 days in mice injected with a single lethal dose (600 mg/kg) of acetaminophen (APAP), and then treated 2 hours later with 20 mg/kg BPPA.

FIG. 6 illustrates the survivorship and the plasma levels of alanine aminotransferase (ALT) and secretory phospholipase A₂ (sPLA₂) over 14 days in mice injected with a single lethal dose (600 mg/kg) of acetaminophen (APAP), and then treated 4 hours later with 20 mg/kg BPPA.

FIG. 7 illustrates the survivorship over 14 days in mice injected with a single lethal dose (600 mg/kg) of acetaminophen (APAP), and then treated 8 hours later with 20 mg/kg BPPA.

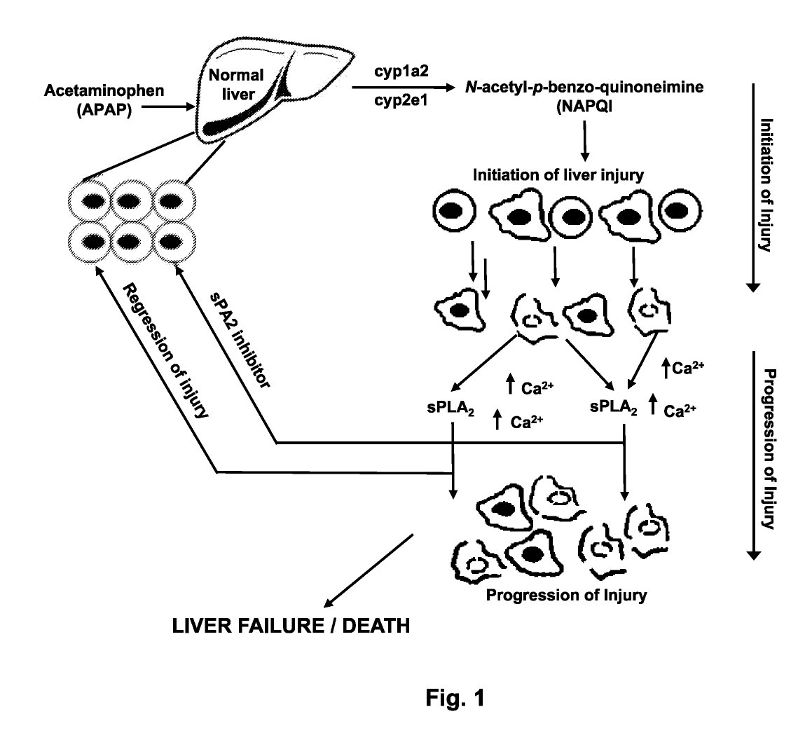

The present invention provides a method of treating or ameliorating the liver damage or mortality due to exposure to high levels of heptotoxicants. I have discovered that if sPLA₂, the death protein involved in the progression of APAP-initiated liver injury is inhibited, it can lead to survival of mice given a lethal dose of a hepatotoxicant. FIG. 1 illustrates the mechanism by which sPLA2 mediates the progression of liver injury. Once a necrogenic event occurs in tissues such as liver, action of death proteins leads to self-perpetuating expansion of injury. Using an overdose of a hepatotoxin, such as acetaminophen, liver injury was stimulated in mice. I found that 60%-80% of mice treated with the near lethal dose (600 mg/kg) of acetaminophen survive if an inhibitor of sPLA₂ (e.g., BPPA) is administered 2 to 8 hours after the drug overdose. Without the inhibitor of sPLA₂, liver samples from the mice given a lethal dose of 600 mg/kg APAP showed progressive expansion of liver injury with time (data not shown). Such a therapeutic rescue of life has not been reported by targeting an enzyme responsible for progression and expansion of liver injury initiated by acetaminophen. In addition to developing a single antidote for saving lives, further development could lead to similar treatment methods for potentially lethal poisoning with other drugs or industrial, occupational, and environmental toxicants.

This new treatment for exposure to liver toxicants targets the death protein and inhibits its activity that would otherwise lead to cell death, rather than targeting the toxin itself. Before sPLA₂ has time to continue tissue destruction, its enzyme activity is inhibited and was shown to stop or slow down the liver destruction. Survivorship increased in mice when treated with an inhibitor of sPLA₂ within 9 hours, preferably within 8 hours, more preferably within 4 hours, and most preferably within 2 hours. Thus the mice could be rescued by administering a single therapeutic treatment with BPPA, well after the poisoning incident. It is also believed that such treatment would be effective to save human patients overdosed with a hepatotoxicant, e.g., acetaminophen.

Secretory phospholipase A₂ (sPLA₂) is a death protein that mediates progression of injury initiated by hepatotoxicants such as acetaminophen (APAP), CCl₄, and thioacetamide. sPLA₂ is normally secreted by various tissue cells and stored in vesicles in tissue cells such as brain, liver, lung, kidney, etc. I have shown that sPLA₂ can be inhibited at 2, 4, and 8 hours after tissue injury was initiated by a lethal dose of a known toxin administered to mice.

As used herein, an inhibitor of secretory phospholipase A₂ refers to any compound that inhibits the activity of sPLA₂ and decreases liver damage to a significant degree when the mammal is exposed to a hepatotoxicant. Many inhibitors are known in the art, including, without limitation, LY329722 (sodium [3-aminooxyalyl-1-benzyl-2-ethyl-6-methyl-1H-indol-4-yloxy]-acetic acid), ochnaflavone, BPPA (5-(4-benzyloxyphenyl)-4S-(7-phenylhepatonoylamino) pentanoic acid, and p-bromophenacylbromide (p-BPB) and other benzophenone oximes derivatized with syndone. Compounds used in the present invention may be administered to a patient by any suitable means, including oral, intravenous, parenteral, subcutaneous, intrapulmonary, and intranasal administration. Parenteral infusions include intramuscular, intravenous, intraarterial, or intraperitoneal administration.

Pharmaceutically acceptable carrier preparations include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampoule, each containing a unit dose amount, or in the form of a container containing multiple doses.

A method for controlling the duration of action comprises incorporating the active compound into particles of a polymeric substance such as a polyester, peptide, hydrogel, polylactide/glycolide copolymer, or ethylenevinylacetate copolymers. Alternatively, an active compound may be encapsulated in nanoparticles or microcapsules by techniques otherwise known in the art including, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

As used herein, an “effective amount” of an inhibitor of sPLA₂ is an amount, that when administered to a patient (whether as a single dose or as a time course of treatment) inhibits or reduces the liver injury due to exposure of a hepatotoxicant to a clinically significant degree; or alternatively, to a statistically significant degree as compared to control. The preferred method of administering the inhibitor is as a single dose. This dose is given within 9 hours of exposure to a hepatotoxicant, preferably within 8 hours, more preferably within 4 hours, and most preferably within 2 hours. “Statistical significance” means significance at the P<0.05 level, or such other measure of statistical significance as would be used by those of skill in the art of biomedical statistics in the context of a particular type of treatment. The term “effective amount” therefore includes, for example, an amount sufficient to decrease the liver damage and/or mortality from exposure to a hepatotoxicant, preferably by at least 50%, and more preferably by at least 90%. The dosage ranges are those that produce the desired effect. Generally, the dosage will vary with the age and condition of the patient, and with the manner of administration. A person of ordinary skill in the art, given the teachings of the present specification, may readily determine suitable dosage ranges. In any event, the effectiveness of treatment can be determined by monitoring symptoms by methods well known to those in the field, for example, monitoring the level of ALT activity.

### EXAMPLE 1

### Materials and Methods

Animals and Diet:

Male Swiss Webster mice weighing between 25-30 g were obtained from Harlan Laboratories (Indianapolis, Ind.). Mice had unlimited access to rodent chow (Harlan Teklad Rat Chow No. 7001, Madison, Wis.) and water. The mice were housed over sawdust bedding free of any known chemical contaminants (Sani-chips; Harlan Teklad, Madison, Wis.) in air conditioned quarters (21±1° C.) with a 12 h photoperiod. All animal husbandry and handling conditions were as per the NIH Guide for Care and Use of Laboratory Animals. Research protocols were approved by the ULM Institutional Animal Care and Use Committee (IACUC).

Treatment Protocol:

Mice were given by intraperitoneal injection (i.p.) a lethal dose of APAP (600 mg/kg in 0.45 N NaCl, pH 8.2) or the vehicle alone. Treated mice were given a single injection of 20 mg/kg, i.p., BPPA, a sPLA₂ inhibitor, after a set time interval (2, 4, and 8 hours after the dose of APAP). BPPA was purchased from a commercial source (Catalog No. 53319, Sigma Aldrich, St. Louis, Mo.). All the mice were observed six times on the first day and twice daily thereafter for 14 days for any mortality. Once every two days, blood was collected from the inferior vena cava in a heparinized container. Plasma was obtained by centrifugation of blood samples (1000 g for 20 min) for assessment of biochemical parameters of liver injury. The remaining plasma was quick-frozen in liquid nitrogen and stored at −80° C. until further analysis.

Hepatotoxicity:

Plasma was separated by centrifugation of the heparinized blood. Plasma alanine aminotransferase (ALT) was measured as a biomarker of liver injury using a kit from Thermo DMA (Waltham, Mass.). The results were ascertained by light microscopic examination of liver sections stained with hematoxylin and eosin (H&E).

sPLA₂ Activity in Plasma and Liver.

sPLA₂ activity in the plasma was measured using sPLA₂ activity kit (Cayman Chemicals, Ann Arbor, Mich.) as per the manufacturer's instructions. sPLA₂ activity in plasma was expressed as sPLA₂ units/ml. One unit of sPLA₂ hydrolyzes one μmole of substrate (diheptanoyl Thio-phosphatidylcholine) per min at 25° C. and is expressed as μmoles substrate hydrolyzed/min/ml.

### EXAMPLE 2

### Normal Levels of ALT and sPLA₂

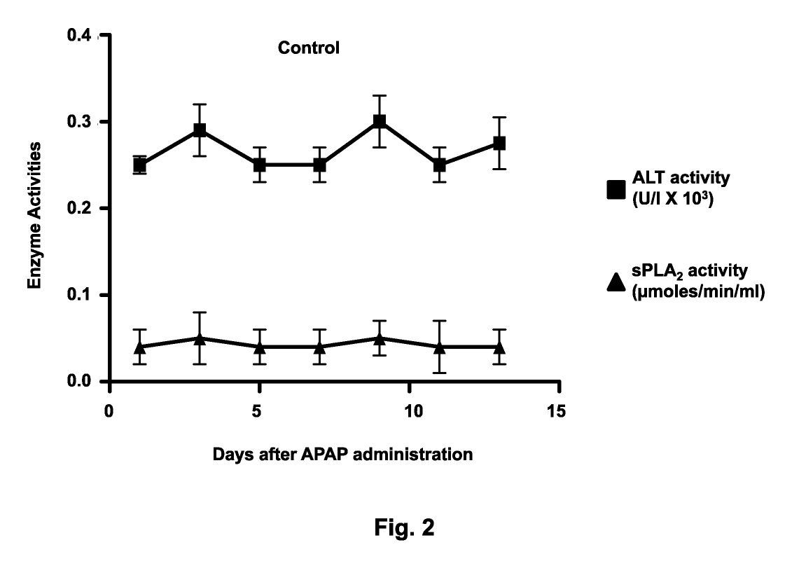

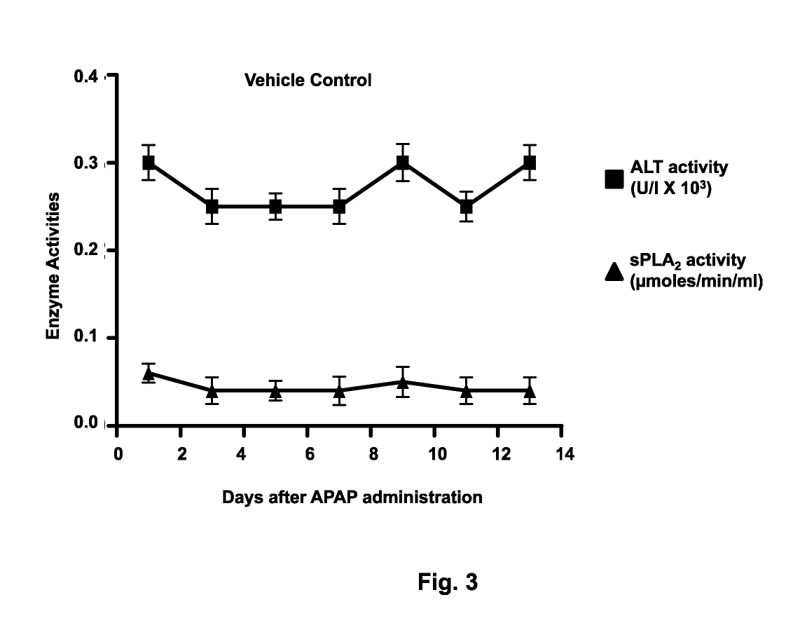

The death protein sPLA₂ and alanine aminotransferase (ALT), a biomarker of liver injury, activities were measured in control and vehicle-treated mice, FIGS. 2 and 3, respectively. Increased activity of plasma sPLA₂ indicates progression of injury, and an increase/decrease in ALT activity indicates increased/decreased liver injury. Ten Male Swiss Webster Mice (30±5 g) were observed as control mice, and were not injected with anything for 14 days. Normal levels of plasma sPLA₂ and ALT activities were measured, and none of the shelf control mice died. The results for ALT and sPLA₂ are shown in FIG. 2.

Another 10 Male Swiss Webster Mice (30±5 g) were treated with vehicle only and observed. The mice were injected with a single dose of warm 0.45 N NaCl given intraperitonealy (i.p.) on Day 1. As shown in FIG. 3, the levels of plasma sPLA₂ and ALT activities in the vehicle-treated mice were similar to the control mice. None of the vehicle-treated mice died, and they appeared healthy and well-groomed.

### EXAMPLE 3

### Treatment of Mice with a Single Lethal Dose of Acetaminophen

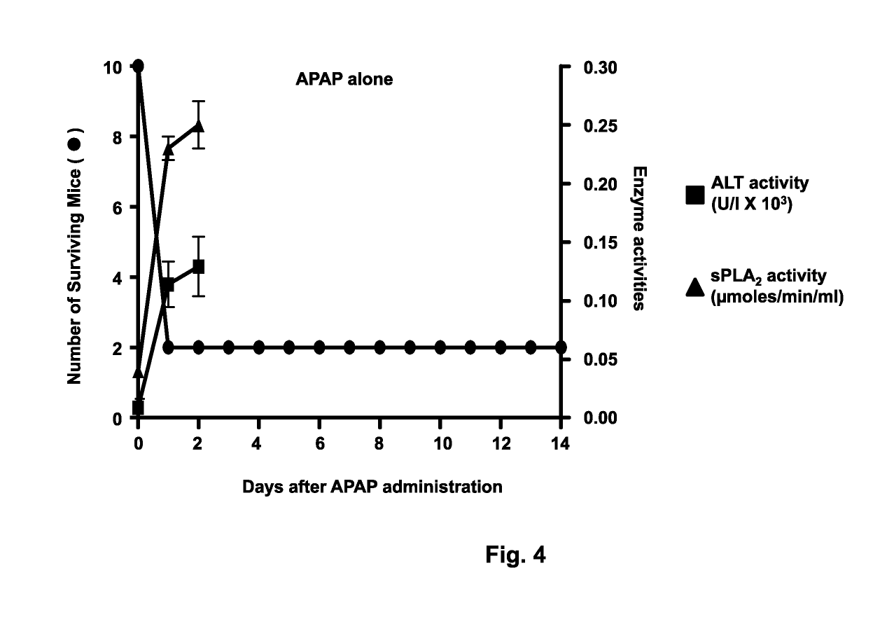

A third group of ten Male Swiss Webster Mice (30±5 g) were injected with a single dose of acetaminophen (APAP), 600 mg/kg, i.p. in 0.45 N NaCl, on Day 1. No further treatment was provided. The enzymatic activity of ALT and sPLA₂ was measured on Day 2. As shown in FIG. 4, the enzymatic activity of both sPLA₂ and ALT increased in these mice, along with significantly decreased survival rates. Administration of a single lethal dose of APAP resulted in 80% mortality. The increased sPLA₂ and ALT activities compared to the control and vehicle treated mice (FIG. 4 as compared to FIGS. 2 and 3) reflect progression of liver injury.

### EXAMPLE 4

### Mice Treated with a Single Lethal Dose of APAP and Injected with BPPA Two Hours Later

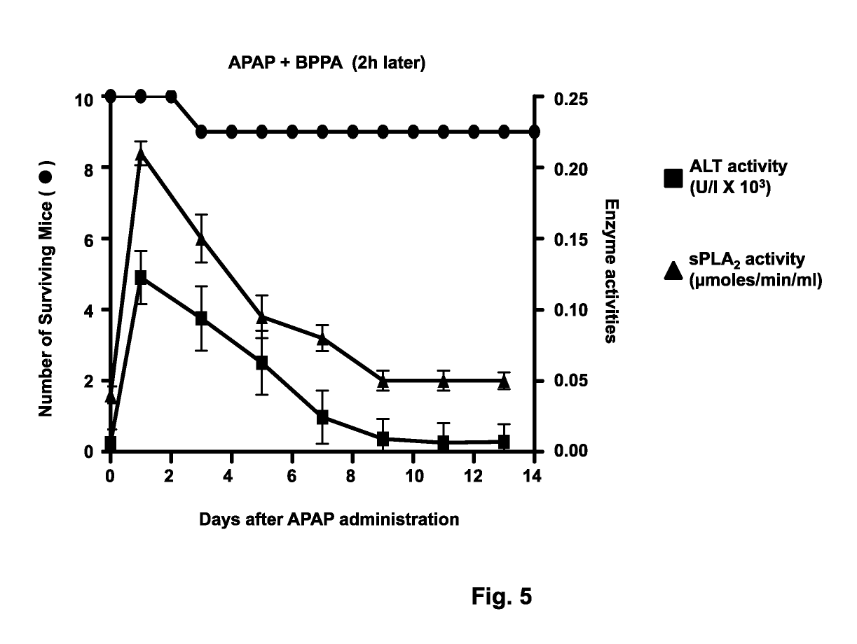

A fourth group of ten Male Swiss Webster Mice (30±5 g) were injected with a single dose of APAP, 600 mg/kg, i.p. in 0.45 N NaCl, on Day 1. Two hours after APAP administration, these mice were injected with 20 mg/kg, i.p., BPPA, a sPLA₂ inhibitor. As shown in FIG. 5, ninety percent of the mice survived when given BPPA (20 mg/kg) administered 2 hours after the lethal dose of APAP. FIG. 5 also shows decreasing levels of sPLA₂ and ALT activities down to the normal values in the surviving mice (FIG. 5 as compared to FIGS. 2 and 3).

### EXAMPLE 5

### Mice Treated with a Single Lethal Dose of APAP Rescued by Injecting BPPA Four Hours Later

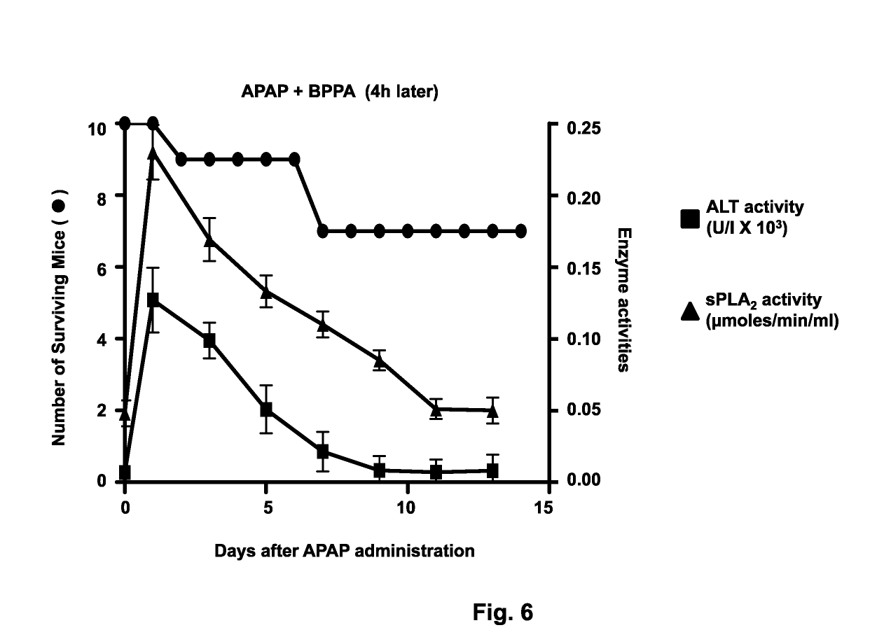

A fifth group of ten Male Swiss Webster Mice (30±5 g) were treated with a single dose of APAP, 600 mg/kg, i.p. in 0.45 N NaCl, on Day 1. Four hours after APAP administration, these mice were injected with 20 mg/kg, i.p., BPPA. As shown in FIG. 6, 70% of the mice survived, and the levels of sPLA₂ and ALT decreased to about normal levels (FIG. 6 as compared to FIGS. 2 and 3).

### EXAMPLE 6

### Mice Treated with a Single Lethal Dose of APAP Rescued by Injecting BPPA Eight Hours Later

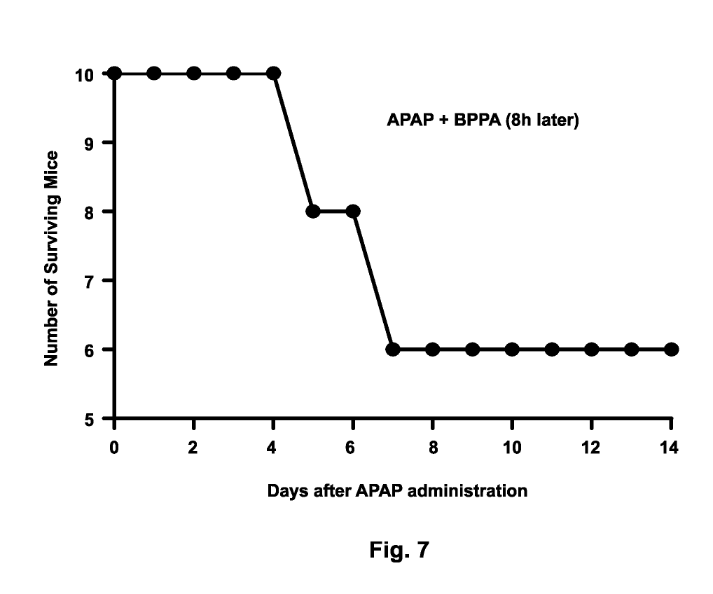

A sixth group of ten Male Swiss Webster Mice (30±5 g) were treated with a single dose of APAP, 600 mg/kg, i.p. in 0.45N NaCl, on Day 1. Eight hours after APAP administration, the mice were injected with 20 mg/kg, i.p., BPPA. As shown in FIG. 7, when BPPA is administered 8 hours after poisoning with a near lethal dose of APAP, 60% of the mice survived. Plasma enzyme levels were not measured in these mice. These findings indicate that mice poisoned with near lethal dose of APAP can be rescued by inhibiting the death protein sPLA₂ as late as 8 hours after poisoning.

Injection of mice with BPPA at different time points after a lethal dose of APAP resulted in a decrease in sPLA₂ and ALT activities indicating decreased progression and extent of liver injury (FIGS. 5 and 6). The survival of mice by injection of BPPA at 2, 4 or 8 hours after APAP administration (FIGS. 5, 6, and 7) indicates that sPLA₂ inhibition leads to prevention of progression of liver injury allowing the survival of the poisoned mice.

Liver injury was initiated in male Swiss Webster mice by a lethal dose of acetaminophen. Survival and mortality were recorded every 12 hours for 14 days. Mortalities were 50%, and 80% at 12 and 24 hours, respectively, after injection of acetaminophen alone (FIG. 4). Fourteen days after the overdose, mortality remained the same. At 14 days, cumulative mortality was only 10% in the mice receiving a single dose of sPLA₂ inhibitor, BPPA (5-(4-benzyloxyphenyl)-4S-(7-phenylheptanoylamino) pentanoic acid) at a dose of 20 mg/kg body weight 2 hours after acetaminophen (FIG. 5). Serum alanine aminotransferase (ALT) and sPLA₂ activities revealed very high liver necrosis and continued progression of liver injury in mice treated with acetaminophen alone (FIG. 4). These enzyme activities decreased in mice receiving BPPA two hours after the administration of acetaminophen (FIG. 5). These findings indicate the destructive role of sPLA₂ in mediating the progression of liver injury initiated by acetaminophen. I have shown that it is possible to inhibit sPLA₂ up to 8 hours after injury and still increase survival rate. Administration of BPPA 4 hours after the administration of acetaminophen resulted in 70% survival rate (FIG. 6). Further, 60% of the mice receiving the lethal dose of acetaminophen survived if BPPA was administered 8 hours after the drug overdose (FIG. 7).

### EXAMPLE 7

### Protection from Other Hepatotoxins Using Inhibitors of sPLA₂

A combination of two chemicals, which are known to be nontoxic individually at very low levels, is known to be highly toxic when combined in mammals. One of these is a chlorinated pesticide known as KEPONE™, which is commonly known as chlordecone. The other is the organic solvent, carbon tetrachloride (CCl₄). Either chemical will be toxic if given at a sufficient dose. For example, chlordecone, administered to older rats at 120 mg/kg causes central nervous system (CNS) toxicity such as tremors, convulsions and death. CCl₄ injected or given orally at 4 ml/kg causes severe liver necrosis, liver failure and death. Studies have shown that exposing rats to 10 ppm chlordecone in a powdered diet for 15 days did not cause any toxicity. Likewise, exposing rats to a single dose of 100 μl/kg CCL₄, i.p. led to the classic centrilobular injury by 3 hours, which escalated to about 4 fold injury in the liver by 12 hours. However, by 24 hours, the liver injury was repaired and no liver injury was observed. In contrast, if the same dose of CCl₄ was given to rats maintained on dietary 10 ppm chlordecone for 15 days, on day 16, all rats died within 3 days after injecting CCl₄, which is about a 67-fold amplification of the toxicity for CCl₄ (Mehendale, 1994).

Using this combination of chlordecone and CCL₄, BPPA (20 mg/kg) was injected in the rats 1 hour after the injection of CCl₄, and 90% of the rats receiving the chlordecone (CD)+CCl₄ survived (data not shown). This indicates that inhibitors of sPLA₂ can also be used to protect from liver injury following a lethal dose of CCL₄.

In addition, I have shown that sPLA₂ mediates the liver injury initiated by thioacetamide, another hepatotoxicant, by showing mice lethally poisoned with thioacetamide were rescued by inhibiting sPLA2 with BPPA (data not shown).

### REFERENCES

[1] Arumugam, T. V., et al. 2003. Comparative protection against rat intestinal reperfusion injury by a new inhibitor of sPLA₂, receptor antagonist. Br. J. Pharmacol. 140, 71-80.
[2] Barbuio, R., Milanski, M., Bertolo, M. B., Saad, M. J., Velloso, L. A. 2007. Infliximab reverses steatosis and improves insulin signal transduction in liver of rats fed a high-fat diet. J. Endocrinol. 194, 539-550.
[3] Bhave, V. S., Donthamsetty, S., Latendresse, J. R., Mehendale, H. M. 2008a. Inhibition of cyclooxygenase-2 aggravates secretory phospholipase A₂-mediated progression of acute liver injury. Toxicol. Appl. Pharmacol. 228, 239-246.
[4] Bhave, V. S., Donthamsetty, S., Latendresse, J. R., Muskhelishvili, L., Mehendale, H. M. 2008b. Secretory phospholipase A₂ mediates progression of acute liver injury in the absence of sufficient cyclooxygenase-2. Toxicol. Appl. Pharmacol. 228, 225-238.
[5] Chanda, S., Mangipudy, R. S., Warbritton, A., Bucci, T. J., Mehendale, H. M. 1995. Stimulated hepatic tissue repair underlies heteroprotection by thioacetamide against acetaminophen-induced lethality. Hepatology 21, 477-486.
[6] Chanda, S., Mehendale, H. M. 1995. Nutritional impact on the final outcome of liver injury inflicted by model hepatotoxicants: effect of glucose loading. FASEB J. 9, 240-245.
[7] Chilakapati, J. et al. 2007. Role of CYP2E1 and saturation kinetics in the bioactivation of thioacetamide: effects of diet restriction and phenobarbital. Toxicol. Appl. Pharmacol. 219, 72-84.
[8] Chilakapati, J. et al. 2007. Toxicokinetics and toxicity of thioacetamide sulfoxide: a metabolite of thioacetamide. Toxicology 230, 105-116.
[9] Cunningham, T. J. et al. (2004) Systemic treatment of cerebral cortex lesions in rats with new secreted phospholipase A₂ inhibitor. J. Neurotrauma 21, 1683-1691
[10] Donthamsetty, S., Bhave, V. S., Mitra, M. S., Latendresse, J. R., Mehendale, H. M. 2008. Nonalcoholic steatohepatitic (NASH) mice are protected from higher hepatotoxicity of acetaminophen upon induction of PPAR alpha with clofibrate. Toxicol. Appl. Pharmacol. 230, 327-337.
[11] Dzavik, V., et al. 2010. The sPLA₂ inhibition to decrease enzyme release after percutaneousl coronary intervention (SPIDER-PCI) trial. Circulation. 122, 2411-2418.
[12] Fuijoka, D., et al. 2008. Reduction in myocardial ischemia/reperfusion injury in group X secretory phospholipase A2-deficient mice. Circulation. 117, 2977-2985.
[13] Gilroy, D. W., Newson, J., Sawmynaden, P., Willoughby, D. A., Croxtall, J. D. 2004. A novel role for phospholipase A₂ isoforms in the checkpoint control of acute inflammation. FASEB J. 18, 489-498.
[14] Hayami, S. et al. (1999) Increase of caspase-3 activity in rat liver and plasma by thioacetamide. Biochem. Pharmacol. 58, 1941-1943
[15] Ishida, Y., Kondo, T., Tsuneyama, K., Lu, P., Takayasu, T., Mukaida, N. 2004. The pathogenic roles of tumor necrosis factor receptor p55 in acetaminophen-induced liver injury in mice. J. Leukoc. Biol. 75, 59-67.
[16] Ivandic, B. et al. (1999) Role of group II secretory phospholipase A₂ in atherosclerosis: 1. Increased atherogenesis and altered lipoproteins in transgenic mice expressing group IIa phospholipase A₂. Arterioscler. Thromb. Vasc. Biol. 19, 1284-1290.
[17] Kamble, R. R., et al. 2009. Synthesis and evaluation of benzophenone oximes derivatized with sydnone as inhibitors of secretory phospholipase A2 with anti-inflammatory activity. Chem. Pharm. Bull. (Tokyo), 57, 16-21.
[18] Karakas, M., et al. 2009. Varespladib methyl, an oral phospholipase A2 inhibitor for the potential treatment of coronary artery disease. Drugs. 12, 585-592.
[19] Kawabata, K., Fujioka, D., Kobayashi, T., Saito, Y., Obata, J. E, Nakamura, T., Yano, T., Watanabe, K., Watanabe, Y., Mishina, H., Kugiyama, K. 2010. Inhibition of secretory phospholipase A2 activity attenuates acute cardiogenic pulmonary edema induced by isoproterenol infusion in mice after myocardial infarction. J. Cardiovasc. Pharmacol. 56, 369-78.
[20] Kramer, R. M. et al. (1989) Structure and properties of a human nonpancreatic phospholipase A₂. J. Biol. Chem. 264, 5768-5775.
[21] Larson, A. M., Polson, J., Fontana, R. J., Davern, T. J., Lalani, E., Hynan, L. S., Reisch, J. S., Schiodt, F. V., Ostapowicz, G., Shakil, A. O., Lee, W. M. 2005. Acetaminophen-induced acute liver failure: results of a United States multicenter, prospective study. Hepatology 42, 1364-1372.
[22] Limaye, P. B., Apte, U. M., Shankar, K., Bucci, T. J., Warbritton, A., Mehendale, H. M. 2003. Calpain released from dying hepatocytes mediates progression of acute liver injury induced by model hepatotoxicants. Toxicol. Appl. Pharmacol. 191, 211-226.
[23] Limaye, P. B., Bhave, V. S., Palkar, P. S., Apte, U. M., Sawant, S. P., Yu, S., Latendresse, J. R., Reddy, J. K., Mehendale, H. M. 2006. Upregulation of calpastatin in regenerating and developing rat liver: role in resistance against hepatotoxicity. Hepatology, 379-388.
[24] Malleo, G., Mazzon, E., Siriwardena, A. K., Cuzzocrea, S. 2007. Role of tumor necrosis factor-alpha in acute pancreatitis: from biological basis to clinical evidence. Shock 28, 130-140.
[25] Mangipudy, R. S. et al. (1995) Tissue repair response as a function of dose in thioacetamide toxicity. Environ. Health Persp. 103, 260-267.
[26] Mehendale, H. M. 1991. Role of hepatocellular regeneration and hepatolobular healing in the final outcome of liver injury. A two-stage model of toxicity. Biochem. Pharmacol. 42, 1155-1162.
[27] Mehendale, H. M. 1994. Amplified interactive toxicity of chemicals at nontoxic levels: mechanistic considerations and implications to public health. Environ. Health Persp. 102 (Suppl. 9), 139-149.
[28] Mehendale H M, Limaye P B. 2005. Calpain: a death protein that mediates progression of liver injury. Trends Pharmacol. Sci. 265, 232-236.
[29] Mehendale, H. M. 2007. Role of tissue repair in liver injury. In Drug-Induced Liver Disease (N. Kaplowitz, DeLeve, L eds, Ed.). Informa Healthcare, Philadelphia.
[30] Menschikowski, M., Hagelgans, A., Siegert, G. 2006. Secretory phospholipase A₂ of group IIA: is it an offensive or a defensive player during atherosclerosis and other inflammatory diseases? Prostaglandins Other Lipid Mediat. 79, 1-33.
[31] Moon, T. C. et al. 2006. Ochnaflavone, naturally occurring biflavonoid, inhibits phospholipase A₂ dependent phosphatidylethanolamine degradation in a CCL₄-induced rat liver microsome. Biol. Pharm. Bull. 29, 2359-2361.
[32] Murakami, M. et al. (1996) Type II secretory phospholipase A₂ associated with cell surfaces via C-terminal heparin-binding lysine residues augments stimulus-initiated delayed prostaglandin generation. J. Biol. Chem. 271, 30041-30051
[33] Napirei, M., Basnakian, A. G., Apostolov, E. O., Mannherz, H. G. 2006. Deoxyribonuclease 1 aggravates acetaminophen-induced liver necrosis in male CD-1 mice. Hepatology 43, 297-305.
[34] Poli, G. (1993) Liver damage due to free radicals. Br. Med. Bull. 49, 604-620.
[35] Ryvniak, V. V. et al. (1990) Intracellular and extracellular activity of cathepsin D in the liver in cirrhosis and its involution. Bull. Eksp. Biol. Med. 109, 199-200.
[36] Sato, R., Yamaga, S., Watanabe, K., Hishiyama, S., Kawabata, K., Kobayashi, T., Fujioka, D., Saito, Y., Yano, T., Watanabe, K., Watanabe, Y., Ishihara, H., Kugiyama, K. 2010 Inhibition of secretory phospholipase A2 activity attenuates lipopolysaccharide-induced acute lung injury in a mouse model. Exp. Lung Res. 36, 191-200.
[37] Shankar, K. et al. (2003) Type 1 diabetic mice are protected from acetaminophen hepatotoxicity. Toxicol. Sci. 73, 220-234
[38] Sun, F. et al. (2000) Evaluation of oxidative stress based on lipid hydroperoxide, vitamin C and vitamin E during apoptosis and necrosis caused by thioacetamide in rat liver. Biochim. Biophys. Acta. 1500, 181-185.
[39] Takasaki, J. et al. (1998) Antibodies against type II phospholipase A₂ prevent renal injury due to ischemia and reperfusion in rats. FEBS Lett. 440, 377-381
[40] Thakore, K. N., Mehendale, H. M. 1991. Role of hepatocellular regeneration in CCl₄ autoprotection. Toxicol. Pathol. 19, 47-58.
[41] Thakore, K. N., Mehendale, H. M. 1994. Effect of phenobarbital and mirex pretreatments on CCl₄ autoprotection. Toxicol. Pathol. 22, 291-299.
[42] Wolf, M. J. et al. (1997) Depletion of intracellular calcium stores activates smooth muscle cell calcium-independent phospholipase A₂. A novel mechanism underlying arachidonic acid mobilization. J. Biol. Chem. 272, 1522-1526.
[43] Woodruff, T. M. et al. (2005) A potent and selective inhibitor of group HA secretory phospholipase A₂ protects rats from TNBS-induced colitis. Int. Immunopharmacol. 5, 883-892.

The complete disclosures of all references cited in this application are hereby incorporated by reference. Specifically incorporated by reference are the following references: (1) Mehendale, H. M. (2012). Once initiated, how does toxic liver injury expand? Trends in Pharmacological Sciences, vol. 33, 200-206; and (2) Bhave, V. S., Domthamsetty, S., Latendresse, J. R., Cunningham, M. L. & Mehendale, H. M. (2011). Secretory phospholipase A₂-mediated progression of hepatotoxicity initiated by acetaminophen is exacerbated in the absence of hepatic COX-2. Toxicology and Applied Pharmacology, 251, 173-180.

What is claimed:

1. A method of protecting or ameliorating the liver damage to mammals exposed to a lethal dose of a hepatotoxicant, said method comprising administering to said mammal an effective amount of an inhibitor of secretory phospholipase A₂ within at least 9 hours of the exposure to the hepatotoxicant, wherein the liver damage is decreased following administration of the inhibitor.

2. The method of claim 1, wherein the hepatotoxicant is acetaminophen.

3. The method of claim 1, wherein the hepatotoxicant is carbon tetrachloride, and wherein the mammal was additionally pre-exposed to chlordecone before exposure to carbon tetrachloride.

4. The method of claim 1, wherein the hepatotoxicant is thioacetamide.

5. The method of claim 1, wherein the inhibitor of secretory phospholipase A₂ is administered within about 2 hours of the exposure to acetaminophen.

6. The method of claim 1, wherein the inhibitor of secretory phospholipase A₂ is administered within about 4 hours of the exposure to acetaminophen.

7. The method of claim 1, wherein the inhibitor of secretory phospholipase A₂ is administered within about 8 hours of the exposure to acetaminophen.

8. The method of claim 1, wherein the inhibitor of secretory phospholipase A₂ is selected from the group consisting of LY329722 (sodium [3-aminooxyalyl-1-benzyl-2-ethyl-6-methyl-1H-indol-4-yloxy]-acetic acid), ochnaflavone, BPPA (5-(4-benzyloxyphenyl)-4S-(7-phenylhepatonoylamino) pentanoic acid, and p-bromophenacylbromide (p-BPB) and other benzophenone oximes derivatized with syndone.

9. The method of claim 1, wherein the inhibitor of secretory phospholipase A₂ is BPPA (5-(4-benzyloxyphenyl)-4S-(7-phenylhepatonoylamino) pentanoic acid.

10. The method of claim 1, wherein the inhibitor of secretory phospholipase A₂ is effective in decreasing liver damage when administered in a single dose.
